(19) United States
(12) Patent Application Publication
(10) Pub. No.: US 2008/0236613 A1
(43) Pub. Date: Oct. 2, 2008

(54) APPARATUS AND METHOD FOR REMOVING DUST FROM OPTICAL LENSES

(75) Inventor: Bor-Yuan Hsiao, Tu-Cheng (TW)

Correspondence Address: PCE INDUSTRY, INC.;ATT. CHENG-JU CHIANG, 458 E. LAMBERT ROAD, FULLERTON, CA, 92835, US

(73) Assignee: HON HAI PRECISION INDUSTRY CO., LTD., Tu-Cheng (TW)

(21) Appl. No.: 11/873,781

(22) Filed: Oct. 17, 2007

(30) Foreign Application Priority Data
Mar. 29, 2007 (CN) 200710200346.6

(51) Int. Cl. B08B 7/02; A61L 2/025

(52) U.S. Cl. 134/1; 422/128; 422/20

(57) ABSTRACT

An exemplary dust removing apparatus applicable for removing dust or dirt from a surface of an optical lens is provided. The dust removing apparatus includes an ultrasonic oscillation device, at least a dust removing device, and a tray. The ultrasonic oscillation device is configured for causing an ultrasonic vibration of the optical lens so as to weaken an adhesive force between the optical lens and the dust on the surface thereof. The dust removing device is configured for removing the dust from the surface of the optical lens. The tray is disposed on the ultrasonic oscillation device and located between the ultrasonic oscillation device and the dust removing device. The tray is configured for supporting the optical lens. A dust removing method is also provided.

### BACKGROUND

1. Technical Field

The present invention relates to dust or dirt removing apparatuses and removing methods, and more particularly, to a removing apparatus and a removing method for removing dust or dirt from a surface of an optical lens.

2. Discussion of Related Art

A lens module generally includes a holder, a lens barrel mechanically engaged with the holder, an image sensor disposed in the holder, and a plurality of optical lenses received in the lens barrel. Due to electro-static effect, dust or dirt may collect on surfaces of the optical lenses during assembly of the lens module. If the dust or dirt collects in the lens barrel, it can cause a degradation of image quality of the lens module.

Therefore, it is necessary to remove the dust or dirt from the surfaces of the optical lenses before the optical lenses are assembled into the lens barrel. Generally, the dust or dirt is manually wiped off by using cotton dipped with alcohol. However, such conventional removing method is time consuming, and the optical lenses may encounter a secondary pollution resulting from the alcohol.

Therefore, what is needed is a dust removing apparatus and a dust removing method which can effectively overcome the above-mentioned drawbacks.

### SUMMARY

A dust removing apparatus applicable for removing dust or dirt from a surface of an optical lens, in associated with a preferred embodiment, is provided. The dust removing apparatus includes an ultrasonic oscillation device, at least a dust removing device, and a tray. The ultrasonic oscillation device is configured (i.e., structured and arranged) for causing an ultrasonic vibration of the optical lens so as to weaken an adhesive force between the optical lens and the dust on the surface thereof. The dust removing device is configured for removing the dust from the surface of the optical lens. The tray is disposed on the ultrasonic oscillation device and located between the ultrasonic oscillation device and the dust removing device. The tray is configured for supporting the optical lens.

A dust removing method configured for removing dust on surfaces of optical lenses, in associated with another preferred embodiment, is provided. The dust removing method includes the following steps: providing a plurality of optical lenses; providing a tray; placing the optical lenses on the tray; applying an ultrasonic vibration to the tray so that an adhesive force between each of the optical lenses and dust thereon is weakened; removing the dust from the surface of each of the optical lenses.

Detailed features of the present dust removing apparatus and dust removing method will become more apparent from the following detailed description and claims, and the accompanying drawings.

### BRIEF DESCRIPTION OF THE DRAWINGS

Many aspects of the present dust removing apparatus and dust removing method can be better understood with reference to the following drawings. The components in the drawings are not necessarily drawn to scale, the emphasis instead being placed upon clearly illustrating the principles of the present dust removing apparatus and dust removing method. Moreover, in the drawings, like reference numerals designate corresponding parts throughout the several views, wherein:

FIG. 1 is a schematic isometric view of a dust removing apparatus, according to a first exemplary embodiment;

FIG. 2 is a cross-sectional view of the dust removing apparatus of FIG. 1, taking along line II-II;

FIG. 3 is a flow-chart of a dust removing method for removing dust on surfaces of optical lenses; and

FIG. 4 is schematic, cross-sectional view of a dust removing apparatus, according to a second exemplary embodiment.

### DETAILED DESCRIPTION OF THE PREFERRED EMBODIMENTS

Reference will now be made to the drawings to describe the preferred embodiments of the dust removing apparatus and the dust removing method, in detail.

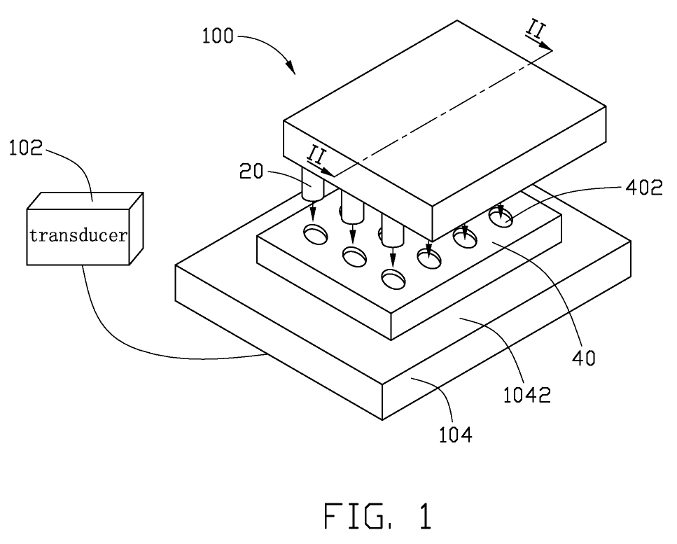

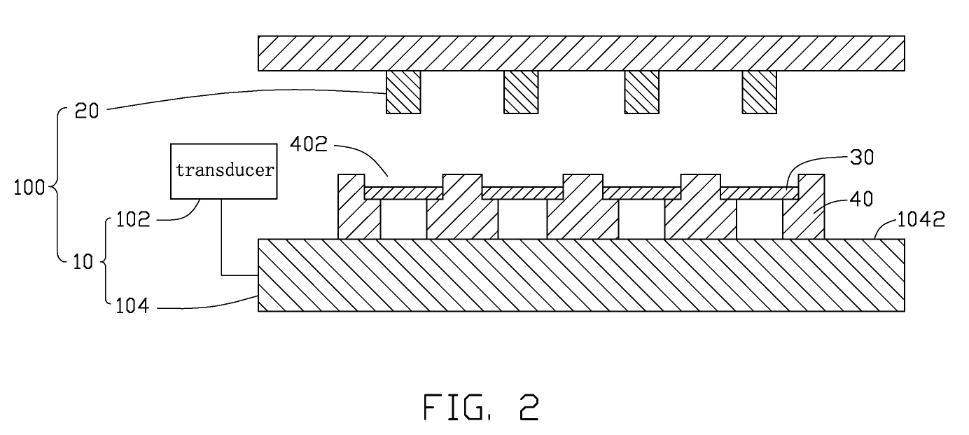

Referring now particularly to FIG. 1 and FIG. 2, a dust removing apparatus 100, according to a first exemplary embodiment of the present invention, is provided. The dust removing apparatus 100 includes an ultrasonic oscillation device 10, a dust removing device 20 and a tray 40.

The ultrasonic oscillation device 10 is configured (i.e., structured and arranged) for causing an ultrasonic vibration of the optical lens so as to weaken an adhesive force between the optical lenses 30 and the dust or dirt on surfaces of the optical lenses 30. The ultrasonic oscillation device 10 includes a transducer 102 and an oscillation transmission member 104 having a radiating surface 1042 in contact with the tray 40. The transducer 102 is configured for converting a pulse voltage form into ultrasonic oscillations. The oscillation transmission member 104 is configured for transmitting the ultrasonic oscillations to the optical lens 30.

The dust removing device 20 is configured for removing the dust from the surfaces of the optical lenses 30. The dust removing device 20 is disposed adjacent to the tray 40 and opposite to the ultrasonic oscillation device 10. In an exemplary embodiment, the dust removing device 20 includes an air blow gun and an ion generator.

The tray 40 is disposed on the radiating surface 1042 and the tray 40 includes a plurality of through holes for receiving optical lenses 30 and a plurality of inner steps formed in the respective through holes for supporting the optical lenses 30 in the through holes.

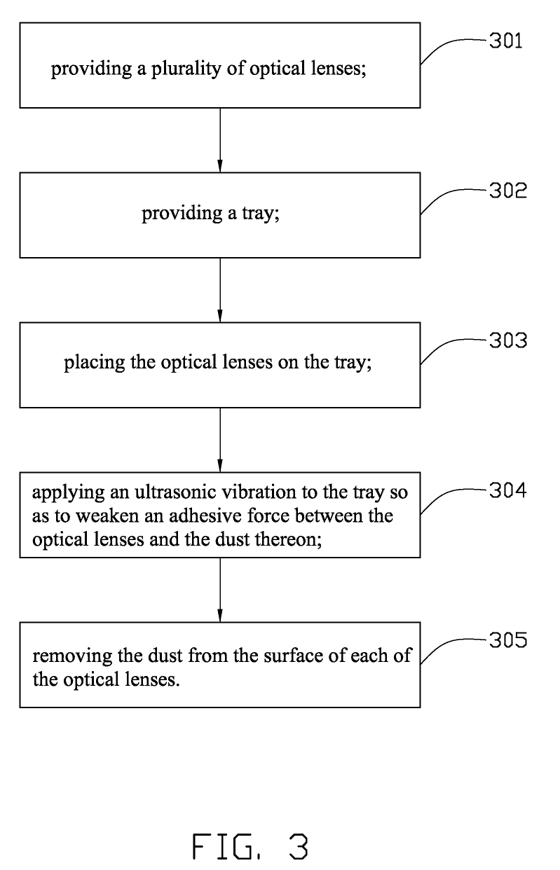

Referring to FIG. 3, a dust removing method for removing dust or dirt on surfaces of optical lenses 30, includes steps as below:
Step 301: providing a plurality of optical lenses 30;
Step 302: providing a tray 40;
Step 303: placing the optical lenses 30 on the tray 40;
Step 304: applying an ultrasonic vibration to the tray 40 so as to weaken an adhesive force between the optical lenses 30 and the dust thereon; and
Step 305: removing the dust from the surface of each of the optical lenses 30.

In step 304, the tray 40 loaded with the optical lenses 30 is located on the radiating surface 1042 of the oscillation transmission member 104, and the dust removing device 20 is disposed adjacent to the tray 40 and opposite to the ultrasonic oscillation device 10. The ultrasonic oscillator 102 drives the oscillation transmission member 104 to vibrate at an ultrasonic frequency, thereby weakening the adhesive force between the optical lens 30 and the dust thereon. The ultrasonic frequency is in a range from 1 megahertz to 10 mega-hertz, and a vibration of the oscillation transmission member 104 lasts for several seconds or several minutes. Preferably, the ultrasonic frequency is 1 megahertz and the vibration of the oscillation transmission member 104 lasts for a time period in a range from 5 seconds to 30 seconds.

In step 305, the dust removing device 20 is started up to remove the dust from the surfaces of the optical lenses 30. The dust removing device 20 can be started up either at the same time when the transducer 102 is started up or after the oscillation transmission member 104 has stopped vibrating.

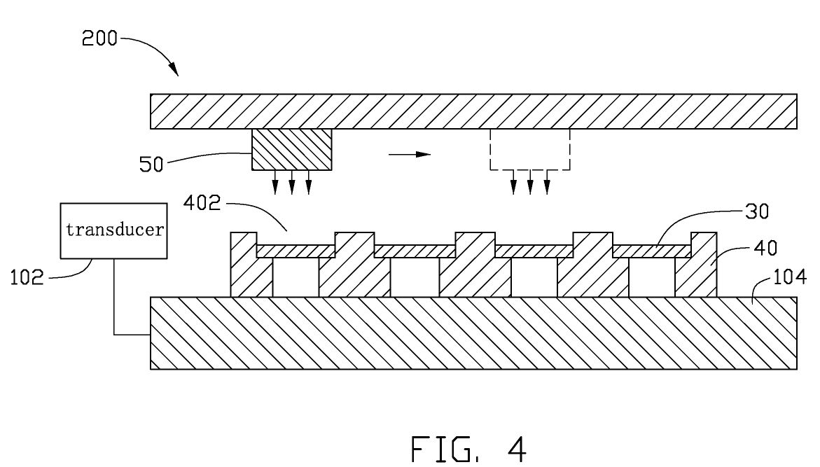

Referring to FIG. 4, a dust removing apparatus 200, according to a second exemplary embodiment of the present invention, is provided. A configuration of the dust removing apparatus 200 is generally similar to the above-mentioned dust removing apparatus 100, and what is different is that the dust removing device 50 of the dust removing apparatus 200 can be powered by a driving device (not shown) to horizontally move so as to more effectively remove the dust on the surfaces of the optical lenses 30.

The dust removing apparatuses 100 and 200 each include an ultrasonic oscillation device 10 and a dust removing device 20, 50, so that can effectively remove the dust or dirt on the surfaces of the optical lenses 30 in batches with high efficiency and without secondary pollution.

Finally, it is to be understood that the above-described embodiments are intended to illustrate rather than limit the invention. Variations may be made to the embodiments without departing from the spirit of the invention as claimed. The above-described embodiments illustrate the scope of the invention but do not restrict the scope of the invention.

What is claimed is:

1. A dust removing apparatus for removing dust from a surface of an optical lens, comprising:
an ultrasonic oscillation device configured for causing an ultrasonic vibration of the optical lens so as to weaken an adhesive force between the optical lens and the dust on the surface thereof;
at least a dust removing device configured for removing the dust from the surface of the optical lens; and
a tray configured for supporting the optical lens, the tray being disposed on the ultrasonic oscillation device and located between the ultrasonic oscillation device and the dust removing device.

2. The dust removing apparatus according to claim 1, wherein the ultrasonic oscillation device comprises a transducer for converting a pulse voltage form into ultrasonic oscillations and an oscillation transmission member in contact with the tray for transmitting the ultrasonic oscillations to the optical lens.

3. The dust removing apparatus according to claim 1, wherein the dust removing device comprises an air blow gun and an ion generator.

4. The dust removing apparatus according to claim 1, wherein the tray comprises a plurality of through holes for receiving optical lenses and a plurality of inner steps formed in the respective through holes for supporting the optical lenses in the through holes.

5. A dust removing method for removing dust on surfaces of optical lenses, comprising steps:
providing a plurality of optical lenses;
providing a tray;
placing the optical lenses on the tray;
applying an ultrasonic vibration to the tray so as to weaken an adhesive force between each of the optical lenses and the dust thereon; and
removing the dust from the surface of each of the optical lenses.

6. The dust removing method according to claim 5, wherein the ultrasonic vibration is generated by an ultrasonic oscillation device, the ultrasonic oscillation device comprising a transducer and an oscillation transmission member, the tray in contact with the oscillation transmission member.

7. The dust removing method according to claim 5, wherein the dust is removed using an air blow gun and an ion generator.

8. The dust removing method according to claim 5, wherein a frequency of the ultrasonic vibration is in a range from 1 mega-hertz to 10 mega-hertz.

9. The dust removing method according to claim 5, wherein the ultrasonic vibration lasts for a time period in a range from 5 seconds to 30 seconds.
